(19) United States
(12) Patent Application Publication
(10) Pub. No.: US 2026/0102277 A1
(43) Pub. Date: Apr. 16, 2026

(54) DIP MOLDED ARTICLE WITH LOCALIZED REINFORCEMENT

(71) Applicant: Church & Dwight Co., Inc., Princeton, NJ (US)

(72) Inventors: Jon Toliver, Pennington, NJ (US); Timothy Snowden, Howell, NJ (US); Salima Hammadi, Somerville, NJ (US); Kaleem Ahmad, Princeton, NJ (US)

(73) Assignee: Church & Dwight Co., Inc., Princeton, NJ (US)

(21) Appl. No.: 19/354,163

(22) Filed: Oct. 9, 2025

Related U.S. Application Data
(60) Provisional application No. 63/705,730, filed on Oct. 10, 2024.

(51) Int. Cl. A61F 6/04

(52) U.S. Cl. CPC A61F 6/04

(57) ABSTRACT

The present disclosure provides a condom that includes an elongated tube defined by an outer wall that includes an elastomeric material. The elongated tube may have a closed end and an open end. The elongated tube may have a tip portion that extends a distance from the closed end toward the open end and a shaft portion that extends from the open end to the tip portion. The outer wall in the tip portion has a tip portion thickness, and the outer wall in the shaft portion has a shaft portion thickness. A ratio of the tip portion thickness to the shaft portion thickness (T/S) is about 1.5 or greater.

### CROSS REFERENCE TO RELATED APPLICATIONS

The present application claims priority to and the benefit of U.S. Provisional Patent Application No. 63/705,730, filed Oct. 10, 2024. The entire contents of which are incorporated herein by reference.

### FIELD OF THE DISCLOSURE

The present disclosure relates to condoms and, more particularly, to condoms having varying thicknesses in the outer wall along the length of the condom.

### BACKGROUND

Condoms and other prophylactics generally have an elongated tubular shape with an open end and a closed end, which may provide a physical barrier against the transmission of bodily and other fluids when in use. The closed end of a condom is often referred to as a tip portion, and the remainder of the length of the condom is referred to as a shaft portion. Typically, condoms are formed of layers of latex, and may have a uniform thickness along the length of the condom. If the condom is too thin, the condom may risk rupturing during use. If the condom is thick enough to reduce the risk of rupture, the sensation for both users may be decreased during use.

The use of texture on the outer surface of a condom, such as ribbing, has been previously suggested and commercialized in order to provide stimulation to the users. This typically only impacts the female experience during use. There remains a desire to reinforce the tip of the condom while keeping the shaft thin to provide the reassurance of safety and protection without compromising sensation between partners during intercourse.

### SUMMARY OF THE DISCLOSURE

The present disclosure is directed to a condom having a varied thickness along the length of the condom. Specifically, the condom may have a thickened tip portion and a thinner shaft portion.

In one or more embodiments, the present disclosure can relate to a condom having an elongated tube defined by an outer wall. The condom may be composed of an elastomeric material. The elongated tube has a closed end and an open end. The elongated tube may have a tip portion that extends a distance from the closed end toward the open end, and a shaft portion that extends from the open end toward the tip portion. The outer wall in the tip portion has a tip portion thickness, and the outer wall in the shaft portion has a shaft portion thickness. A ratio of the tip portion thickness to the shaft portion thickness (T/S) can be, for example, about 1.5 or greater.

In some embodiments, the ratio of the tip portion thickness to the shaft portion thickness (T/S) may range from about 1.6 or greater or may range from about 1.6 to about 3.8.

In some embodiments, the tip portion thickness ranges from about 75 μm to about 150 μm, or from about 100 μm to about 150 μm, or from about 125 μm to about 150 μm. The shaft portion thickness may range from about 40 μm to about 95 μm.

In one or more embodiments, the condom may include or have layers of an elastomeric material. A number of the layers defining the outer wall in the shaft portion may be a whole number (n) of layers of the elastomeric material. The number of layers defining the outer wall in the tip portion may be a whole number (x) of the same elastomeric material or a different elastomeric material, wherein x is greater than n. In some embodiments, a sum of n and x may be 2 or greater, and may range from, for example, 2 to 5.

In some embodiments, a distance the tip portion may extend from the closed end of the condom and may be about 5% to about 50% of a total length of the condom, or the distance may be about 10% to about 25% of the total length of the condom.

In one or more embodiments, the condom may include or have layers of an elastomeric material. The outer wall of the elongated tube can include one or more full-length layers and one or more partial-length layers. A full-length layer of the plurality of full-length layers may have a length of at least 180 mm, and a partial-length layer of the plurality of partial-length layers may have a length ranging from about 10 mm to about 90 mm.

In one or more embodiments, the outer wall of the elongated tube may have a first layer, a second layer, and a third layer. As an example, the first layer may include a full-length layer, the second layer may include a partial-length layer, and the third layer may include a full-length layer. As another example, the first layer may include a partial-length layer, the second layer may include a full-length layer, and the third layer may include a full-length layer. As a further example, the first layer may include a full-length layer, the second layer may include a full-length layer, and the third layer may include a partial-length layer.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1 is a schematic of a condom having an elongated tube defined by an outer wall, a closed end, an open end, a tip portion, a shaft portion, a tip portion length, and a shaft portion length.

FIG. 2A is an image of a condom produced using a three-dip molding process, with the third dip being a partial dip, and FIG. 2B is an image of the partially dipped region, corresponding to the tip portion, that has been magnified where the arrows indicate length of the partial dip defining the edge of the tip portion.

FIG. 3A is an image of a condom produced with partial dip defining a tip portion length of 14 mm from a closed end using pigmented latex for the partial dip, FIG. 3B is an image of a condom produced with partial dip defining a tip portion length of 26 mm from a closed end using pigmented latex for the partial dip, and FIG. 3C is an image of a condom produced with partial dip defining a tip portion length of 56 mm from a closed end using pigmented latex for the partial dip.

FIG. 4 is an image of a condom having three layers, arranged as a first, full length layer, a third, full length layer, and a second, partial dip layer positioned between the first layer and the second layer, where the second, partial dip layer forms the colored tip portion.

FIG. 5 is a graph showing the statistical significance of the mean vibration threshold values in human subjects compared between skin and the physically blocking of sensation using films at increasing thicknesses of approximately 40 μm, 70 μm, and 140 μm as described in Example 4.

### DETAILED DESCRIPTION OF THE DISCLOSURE

The invention now will be described more fully herein after through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The term “about” or “approximately” are defined as being close to as understood by one of ordinary skill in the art. In one non-limiting embodiment, the terms are defined to be within 10%, preferably within 5%, more preferably within 1%, and most preferably within 0.5%. The terms “removing,” “removed,” “reducing,” “reduced,” or any variation thereof, when used in the claims and/or the specification includes any measurable decrease of one or more components in a mixture to achieve a desired result. The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The term “plurality” as used herein refers to two or more items or components. The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The present disclosure relates to a condom having at least one layer of an elastomeric material along the entire length of the condom and having at least one additional layer of an elastomeric material only at the tip portion or closed end of the condom.

Condoms are physical barrier prophylactic device worn over a penis and used during intercourse to reduce the probability of pregnancy or the transmission of sexually transmitted infections (STI). Condoms are provided in a rolled-form and are put on prior to intercourse. Condoms are typically made from an elastomeric material such rubber latex, polyurethane, polyisoprene, or lamb intestine.

There are several competing features of condoms that may be controlled to impact the user experience and/or effectiveness of the condom. For instance, there is a balance between providing functionality as a prophylactic and also maintaining sensitivity for the user. As an example, it may be desired to make the walls of the condom thin to promote sensitivity of the users; however, if the walls of the condom are too thin the condom may rupture during use, rendering it ineffective at preventing pregnancy or STI transmission. Conversely, if the walls of the condom are too thick, which is advantageous in preventing rupture of the condom during use, the thickness may cause a loss of sensation for both users during intercourse which is an undesirable effect.

Attempts to address this issue have been made in commercially available condoms by providing texturing onto the surface of the condom. For example, “ribbed” condoms provide a conventional shaped condom that may have a raised pattern on the outer or inner surface of the condom to provide additional stimulation to the users during intercourse. However, such texturing or raised patterning may cause irritation during use. The present disclosure thus provides condoms that are configured to provide adequate protection, while also enhancing sensitivity for the users.

In one or more embodiments, the present disclosure provides a condom having multiple layers of elastomeric materials, where at least one layer of the elastomeric material is only present at the closed end of the condom, which covers the tip of the penis during use of the condom. This closed end region may be referred to as a “tip portion” or “tip region.” The layer of the elastomeric material that is present only at the tip portion of the condom provides reinforcement of the tip of the condom while keeping the shaft portion of the condom thin. This provides the right balance of maintaining safety and protection without compromising sensation between partners during intercourse.

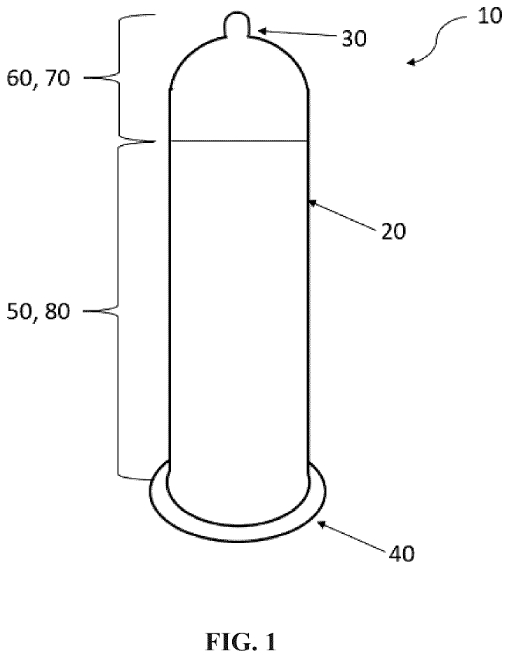

As shown in FIG. 1, condom 10 may include an elongated tube defined by an outer wall 20 that may include an elastomeric material. The elongated tube has a closed end 30 and an open end 40. The elongated tube may have a tip portion 60 that extends a distance from the closed end 30 toward the open end 40 and a shaft portion 50 that extends from the open end 40 to the tip portion 60. The tip portion 60 may have a tip portion length 70, and the shaft portion may have a shaft portion length 80.

In one or more embodiments, the thickened tip portion may be effective to decrease sensation at the tip of the glans penis and also increase sensations near the frenulum of prepuce on the underside of the glans penis. The frenulum is responsive to touch which may be stimulated more when using a condom with an additional layer of the elastomeric material. The thickened elastomeric material provides a more rigid region of the condom near the frenulum, and the thinner latex provides a softer region which may move during intercourse and continually stimulate this area. In addition to increased sensations under the glans penis, the thinner latex of the design allows more sensation exchanged between partners during intercourse along the shaft. Having a more reinforced tip may also make the feature more prominent when the condom is in the rolled-up prior state to use, which may aid in the proper orientation of the condom during donning and aid in the process of putting the condom on. Having a distinctly thickened tip portion or region of the condom and thinner shaft may provide more comfort in use via a more flexible film for the shaft of the penis and smaller diameter for the rolled ring bead once unrolled.

In embodiments of the condom, the thickness of the tip portion of the condom and the thickness of the shaft portion of the condom may be different. The condom has an overall shape of an elongated tube or tubular shaft that has a closed end and open end. The open end receives the user's penis upon putting the condom on prior to use. The elongated tube has an outer wall. Specifically, the “thickness” of the tip portion or shaft portion refers to the thickness of the outer wall of the condom at the respective tip or shaft portion of the condom. The thickness of the outer wall of the condom may be controlled during the process of manufacturing the condom.

In one embodiment, the condom may be manufactured using a dip molding process, where elongated tube-shaped mandrels are dipped in a bath that contains an elastomeric material mixture, followed by curing. The number of the dips may be modified to form layers on the mandrel. The length of the mandrel dipped into the bath may also be modified. For example, in a three-dip molding process, two dips may be performed for the entire length of the mandrel to form two layers of the elastomeric material extending along the full length of the condom. The third dip into the bath may be a partial dip and may be carried out prior to the forming the first full length layer, between form the first full length layer and the second full length layer, or after forming both of the first full length layer and the second full length layer. Additional dips may be added to suitably alter the length and thickness of the partial dip region of the condom. This process of partially dipping the mandrel forms additional layers at the tip portion of the condom. This process may also be performed by spraying, altering the order of dipping, or utilizing techniques including coagulant dipping. Coagulant dipping is a straight condom dipping process in which a destabilizing chemical (i.e., the coagulant) is coated on the condom elongated tube-shaped mandrel. When the mandrel is immersed into the latex bath, the coagulant reacts with the colloid stabilizer on the latex in the immediate vicinity of the mandrel. Typically, the amount of latex coated on the mandrel from a coagulant dipping process is much higher than when dipping an uncoated mandrel into a latex bath.

The condom may have a number of layers that define the outer wall of the condom formed using a dip molding process. In some embodiments, the number of the layers in the shaft portion may be a whole number (n) of layers of the elastomeric material. The number of layers of the outer wall of the tip portion may be a whole number (x) of the same elastomeric material or a different elastomeric material. In some embodiment, the number of layers in the tip portion, x, is greater than the number of layers in the shaft portion, n. This indicates that the thickness of the tip portion is greater than the thickness of the shaft portion of the condom. More specifically, a total number, or sum of n and x, of the layers of the tip portion and the shaft portion may be 2 or greater, or may range from 2 to 7, 2 to 6, 2 to 5, 2 to 4, 2 to 3, 3 to 7, 3 to 6, 3 to 5, 3 to 4, 4 to 7, 5 to 7, 6 to 7, 4 to 6, or 5 to 6.

In some embodiments, the condom may have one or a plurality of full-length layers and one or a plurality of partial-length layers. A full-length layer corresponds to a layer extending from the closed end of the condom to the open end of the condom. A partial-length layer corresponds to a layer extending from the closed end of the condom to a partial length toward the open end of the condom and does not extend fully to the open end of the condom. The partial-length layer is formed using a partial dip during the molding process by dipping the mandrel into the bath containing the elastomeric mixture, the dipping being a distance that is less than the full length of the mandrel, such as less than 50%, less than 40%, less than 30%, or less than 25% of the total length of the mandrel. By including a partial-length layer in the outer wall of the condom, the tip portion may be thickened in comparison to the shaft portion.

There may be a variety of configurations of the full-length layers and partial-length layers. For example, in a condom having 3 layers, the first layer may be a full-length layer, the second layer may be a partial length layer, and the third layer may be a full-length layer.

A full-length layer may have a length of at least about 180 mm, about 180 mm to about 220 mm, about 185 mm to about 215 mm, about 190 mm to about 210 mm, or about 200 mm to about 205 mm. A partial-length layer may have a length ranging from about 10 mm to about 90 mm, about 15 mm to about 85 mm, about 20 mm to about 80 mm, about 25 mm to about 75 mm, about 30 mm to about 70 mm, about 35 mm to about 65 mm, about 40 mm to about 60 mm, or about 45 mm to about 55 mm.

The tip portion or tip region of the condom may have an outer wall having a thickness that may range from about 75 μm to about 150 μm, about 85 μm to about 150 μm, about 100 μm to about 150 μm, about 115 μm to about 150 μm, about 125 μm to about 150 μm, about 75 μm to about 115 μm, about 75 μm to about 100 μm, or about 75 μm to about 85 μm.

The shaft portion or shaft region of the condom may have an outer wall having a thickness that may range from about 40 μm to about 95 μm, about 45 μm to about 90 μm, about 50 μm to about 85 μm, about 55 μm to about 80 μm, about 60 μm to about 75 μm, about 65 μm to about 75 μm, about 65 μm to about 85 μm, about 70 μm to about 80 μm, or about 65 μm to about 70 μm.

A T/S ratio, which is a ratio of the tip portion thickness (T) to the shaft portion thickness(S), may range from about 0.3 or greater, about 0.3 to about 3.8, about 0.5 to about 3.6, about 0.7 to about 3.4, about 0.9 to about 3.2, about 1.1 to about 3.0, about 1.1 to about 2.8, or about 1.3 to about 2.6, in some embodiments. In preferred embodiments, the T/S ratio may range from about 1.5 or greater, about 1.5 to about 3.8, about 1.6 to about 3.8, about 1.7 to about 3.8, about 1.8 to about 3.8, about 1.9 to about 3.8, about 2.0 to about 3.8, about 2.1 to about 3.8, about 2.2 to about 3.8, about 2.3 to about 3.8, about 2.4 to about 3.8, about 2.5 to about 3.8, about 2.6 to about 3.8, about 2.7 to about 3.8, about 2.8 to about 3.8, about 2.9 to about 3.8, about 3.0 to about 3.8, about 3.1 to about 3.8, about 3.2 to about 3.8, about 3.3 to about 3.8, about 3.4 to about 3.8, about 3.5 to about 3.8, about 3.6 to about 3.8, or about 3.7 to about 3.8. The ratio of the tip portion to the shaft portion within these ranges is effective to provide improved stimulation to user of the condom during intercourse.

The tip portion or tip region of the condom extends from a closed end of the condom toward the open end of the condom. A distance that the tip portion extends from the closed end may be about 5% to about 50% of a total length of the condom, about 10% to about 45% of a total length of the condom, about 15% to about 40% of a total length of the condom, about 20% to about 35% of a total length of the condom, about 25% to about 30% of a total length of the condom, about 10% to about 25% of a total length of the condom, about 15% to about 30% of a total length of the condom, or about 5% to about 25% of a total length of the condom.

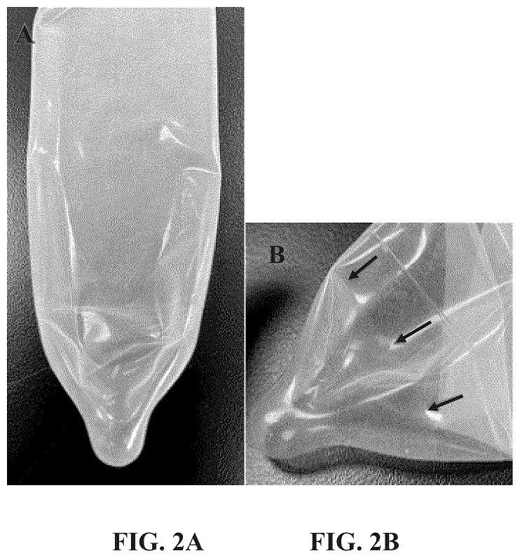

For example, FIGS. 2A and 2B show an image of an example of a condom made using a three-dip molding process described in Example 1. FIG. 2A is an image showing the tip portion and the shaft portion. FIG. 2B is a zoomed in image of the tip portion shown in FIG. 2A with arrows indicating the length of the tip portion. In FIGS. 2A and 2B, the total number of layers is 3.

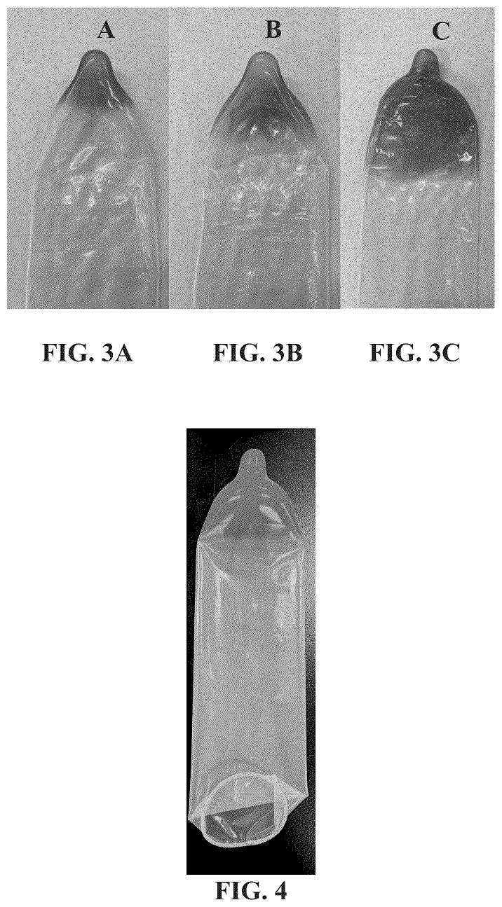

While all three dips can be made with uncolored latex as in FIGS. 2A and 2B, a pigment can be added to the latex for the partial dip region to highlight the tip portion. FIGS. 3A, 3B, and 3C show examples of condoms made with varying third dip lengths using pigmented latex for the partial dip to form the colored tip portions. The shaft portions remain unpigmented.

The pigments present only in the tip portion may include pigments such as red, orange, yellow, green, blue, indigo, and violet, and any intermediate pigment combinations thereof. The presence of color in only the tip portion may result in an overall ombré or gradient coloring effect of the condom, where the tip of the condom is the most pigmented, and the open end of the condom contains no pigment and is “clear” or translucent. The coloring of the tip portion offers users a more fun, unique, vibrant, and exciting experience before and after use. The coloring of the tip portion also acts to conceal or mask seminal fluid present inside the condom after use, which makes the condom less visually unappealing after use.

In some embodiments, the present disclosure may be directed to a method for forming the condom. The method may include steps of forming a first layer of an elastomeric material on a shaped mandrel, forming a second layer of the elastomeric material on a surface of the first layer of the elastomeric layer; and forming a third layer of the elastomeric material on a surface of the second layer of the elastomeric material to form the condom. Between each step, the elastomeric material may be allowed to cure or dry for a specified amount of time, before removing the condom from the shaped mandrel. Each step of forming the condom may include dipping the mandrel into a bath containing a mixture that includes the elastomeric material. The time and length of each dip may be controlled to affect the desired thickness and length of each layer. The resulting condom may have a tubular shaft that includes a thickened tip portion and a thinner shaft portion. The tip portion may extend from a closed-end of the condom toward an opposing open-end of the condom, and the tip portion may have a tip portion thickness that extend a distance from the closed-end of the condom toward the opposing open-end of the condom. The shaft portion may have a shaft portion thickness that extend from the tip portion to the opposing open-end of the condom. A ratio of the tip portion thickness to the shaft portion thickness (T/S) may range from about 1.5 or greater or any other ratio as described herein.

### EXAMPLES

The present disclosure is more fully illustrated by the following examples, which are set forth to illustrate certain embodiments of the present disclosure and are not to be construed as limiting thereof.

### Example 1: Condom Manufactured Using a Three-Dip Molding Process

A condom was manufactured using a three-dip molding process. In the method of manufacturing the condom, glass formers were dipped in a latex bath, and then cured in an oven. Three dips into the latex bath were used. The first two dips were for the entire length of the condom, about 180-200 mm. The third dip was a partial dip with a length about 20 mm from the closed end.

FIG. 2A and FIG. 2B are images of a condom manufactured using the three-dip molding process described above.

### Example 2: Condoms Manufactured Using a Three-Dip Molding Process and Colored Latex

In Example 1, all three dips were made with uncolored latex in the latex bath. A pigment was added to the latex mixture for the partial dip region to highlight the length of the partial dip region. FIGS. 3A-3C show examples of condoms made with varying dip lengths using pigmented latex for the partial dip regions.

FIG. 3A is an image of a condom produced with varying partial dip length of 14 mm from a closed end of the condom. The pigmented latex for the partial dip colored only the tip of the condom. FIG. 3B is an image of a condom produced with varying partial dip length of 26 mm, and FIG. 3C is an image of a condom produced with varying partial dip length of 56 mm. These images demonstrate the thickness of the outer wall of the tip portion of the condom.

### Example 3: Condom Manufactured with Second Layer Having a Partial Dip Between the First Layer and Third Layer

In another embodiment, the second layer was a partial dip and was “sandwiched” between the first and third full-length layers. The condoms with a “sandwiched” partial dip were produced by first dipping the tubular shaped-mandrel for an entire length (about 180 mm to 200 mm) of the condom, followed by a second, partial dip (about 34 mm from the closed end), followed by a third full length dip (about 180 mm to 200 mm). An image of the condom having the thickened tip portion “sandwiched” between the first and third layers is shown in FIG. 4. The tip portion is highlighted with pigmented latex.

### Example 4: Sensitivity Study

The bio-thesiometer® is a device manufactured by Biothesiometer® USA, Newberry, (OH, USA) that includes of a hand-held probe connected to a stationary amplifier, containing an intensity dial and an analog frequency meter. The bio-thesiometer® is used as a research device to assess a range of human sensory perception. Focused investigations have been performed on the innate skin sensitivity of the penile glans and shaft; and observations were also made on the perceived sensations after physical blocking the areas with varied thicknesses of condoms. In general, the amplitude on the instrument may be set to a predetermined level or the amplitude can be increased gradually until the threshold of vibratory sensation is reached. The “threshold of vibratory sensation” refers to a minimum vibration level necessary for an individual panelist to perceive the vibration. This can also be done conversely by lowering the amplitude until the vibration is no longer discernible. In either direction, the amplitude may be determined at any given level with a high degree of accuracy to determine the vibration threshold of a subject.

A bio-thesiometer® as described above was used to conduct a series of sensitivity studies using condoms as manufactured in Examples 1-3 with individuals. First, the individuals participating in the sensitivity study were trained to use bio-thesiometer®. The tip of the bio-thesiometer® device was placed in contact on a body part other than what was tested (i.e., on the wrist) to demonstrate the sensation to be expected. The bio-thesiometer® was dialed up to the maximum voltage so that the individuals could feel the vibration and respond that they felt the vibration. Next, the bio-thesiometer® was dialed down to the minimum voltage, and the individuals confirmed that they no longer felt the vibration. This process was repeated several times to confirm the participants understanding of the range of the vibrations.

Next, the trained individuals tested latex films having various thicknesses. The latex films had thicknesses of approximately 40 μm, 70 μm, and 140 μm as shown in Table 1. Each latex film was placed and secured on the tip of the individuals index finger. The tip of the bio-thesiometer® was applied to the volar pad of each individual's index finger to explore sensory differences associated when varying the film thickness. The vibration intensity of the bio-thesiometer® was slowly increased by turning the voltage dial and was stopped when the individual indicated they felt the vibration and the value was recorded. The vibration intensity was increased, and it was verified that the individual felt the vibration increasing. The vibration intensity was slowly reduced by turning down the voltage dial and was stopped once the individual indicated they no longer felt the vibration. The vibration intensity was recorded. This testing was repeated three times in the same manner with each individual and each different film thickness. Each individual was tested twice with a minimum of 15-minutes in between the testing. An average vibration threshold value was calculated by averaging the two values from each testing.

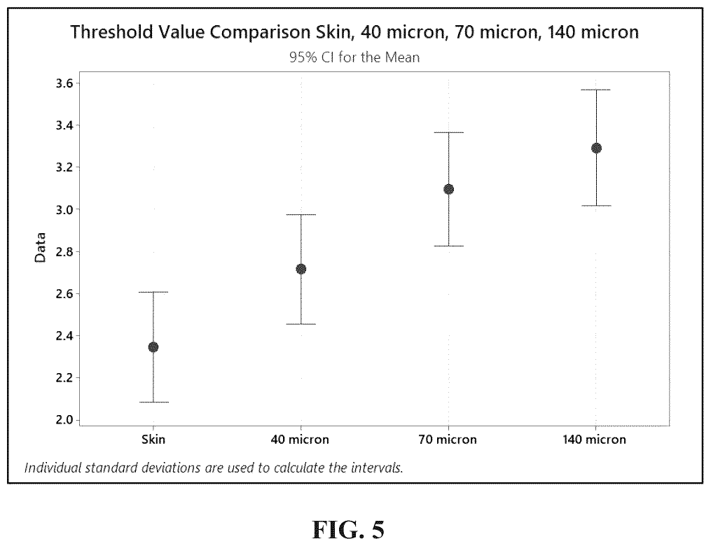

The statistical significance of the average vibration threshold values in human subjects were compared between skin and the physically blocking of sensation using films of increasing thicknesses of approximately 40 μm, 70 μm, and 140 μm as shown in Table 1. The average vibration threshold values were found to increase linearly as the film thickness increased indicating a physical blocking effect to sensation as the pathlength for vibration was increased FIG. 5. Examination of the average vibration threshold values found that adjustments made to the thickness at specific regions on the condom design can deliver variations in the sensation detected by a human subject rather than having a uniform thickness profile along a condom as shown in Table 2. Each condom has a tip portion that has a thickness and a shaft portion that has a thickness. A ratio between the tip thickness (T) to the shaft thickness(S) of the condoms, (T/S), was adjusted between 0.3 to 3.5 to deliver unique sensations in certain areas or a combination of areas. Condoms were manufactured to have a ratio between 0.3 to 3.8, to control the sensations experienced along the glans penis and shaft while using the condom as shown in Table 3. Condoms having a ratio of 1.6 to 3.8 were identified as having improved sensitivity by users.

TABLE 1
Statistical Significance of Mean Threshold Values:
Skin, 40 μm, 70 μm, 140 μm

| Treatment | N | Mean | Grouping |
|---|---|---|---|
| 140 μm | 36 | 3.29167 | A |
| 70 μm | 36 | 3.09722 | B |
| 40 μm | 36 | 2.71528 | C |
| Skin | 36 | 2.34722 | D |

TABLE 2
Ratio of tip thickness to shaft thickness (T/S)
and associated Threshold Values Per region

| | Tip | Tip | Shaft | Shaft | |
|---|---|---|---|---|---|
| | Thickness | Threshold | Thickness | Threshold | |
| Example | (μm) | Value | (μm) | Value | T/S Ratio |
| A | 140 | 3.3 | 40 | 2.7 | 3.5 |
| B | 140 | 3.3 | 70 | 3.1 | 2 |
| C | 140 | 3.3 | 140 | 3.3 | 1 |
| D | 70 | 3.1 | 40 | 2.7 | 1.8 |
| E | 70 | 3.1 | 70 | 3.1 | 1 |
| F | 70 | 3.1 | 140 | 3.3 | 0.5 |
| G | 40 | 2.7 | 40 | 2.7 | 1 |
| H | 40 | 2.7 | 70 | 3.1 | 0.6 |
| I | 40 | 2.7 | 140 | 3.3 | 0.3 |

TABLE 3
Ratio of tip thickness to shaft thickness

| | | T/S |
|---|---|---|
| Tip thickness (μm) | Shaft Thickness (μm) | Ratio |
| 150 | 40 | 3.8 |
| 150 | 75 | 2.0 |
| 75 | 40 | 1.9 |
| 150 | 95 | 1.6 |
| 130 | 80 | 1.6 |
| 130 | 90 | 1.4 |
| 75 | 95 | 0.8 |
| 75 | 150 | 0.5 |
| 40 | 75 | 0.5 |
| 40 | 95 | 0.4 |
| 40 | 150 | 0.3 |

### Example 5: Male Study

A 3½ week in-home sequential monadic study was used to assess three products with a Straight Wall Nipple End (SWNE) design: Commercial Product 1, Commercial Product 2, and Example Product 3. Commercial products 1 and 2 are branded products that are commercially available. Example Product 3 used in the male solo study was a condom having a tip portion and a shaft portion having a (T/S) thickness ratio of 1.6. Commercial Products 1 and 2 each had an average (T/S) ratio of 1.2.

The study consisted of 25 heterosexual cis-gendered males (aged 18-44 years old), condom users, who masturbate at least twice a week. Participants were mailed 3 samples of each product 1-3, and a test kit containing a vaginal simulator, warming sleeve, lubricant, and corn starch. Using the kit items in combination provided a realistic stimulation for feedback on pleasure and comfort during the assessment of currently marketed reference products and present disclosure. The testing panel was trained on the methodology in using a solo approach (no partner) and the items included in the testing kit. Subjects used each product 2-3 times, filling out a diary after each usage occasion, and completing a detailed online questionnaire for each product before moving to the next product. Ten of the respondents also participated in a 45-minute interview at the end of the study to discuss the methodology and their product usage experiences in more detail. Assessments of the performance of the condoms included functional attributes, major likes/dislikes, and preferences.

Based on the variation in the (T/S) ratio, a distinct preference between the tested articles was expressed by the study participants due to the unique sensory experience provided by Example Product 3. The panel rated Example Product 3 the highest (excellent, very good, good) for the level of sensation, intensity of sensation, and liking where (location) they felt the sensations on their penis. There were more varieties in the sensations felt along the shaft of the Example Product 3 design having a (T/S) ratio of 1.6, than the Commercial Products 1 and 2 having a (T/S) ratio of approximately 1.2. The thickened latex in the tip portion of Example Product 3 may have provided a more rigid region of the design and the thinner latex may have provided a softer region that may move during intercourse and continually stimulate the glans and shaft. Additional variations in sensations may be attributed to differences in physically blocking nerves within both regions. The Example Product 3 was also found to have provided better sensation of simulated body heat along the profile. The Example Product 3 was also found to be easy to orientate when donning, and quicker and easier to put on and unroll due to the design.

### Example 6: Partnered Study

Example Product 3 was also tested with couples to gain additional perspective from both male and female users and qualitative insights. A 2½ week sequential monadic home use test was used to assess two condom products with a Straight Wall Nipple End (SWNE) design: Commercial product 1 and Example Product 3. The study consisted of 36 couples (72 individuals). Subjects used each product 2-3 times, filling out a detailed questionnaire for each product before moving to the next product. Nineteen of the respondents were asked to participate in a qualitative interview (either individual discussion interview (IDI), Couple Dyad (two-on-one with a moderator), or mini-focus group (3 to 4 people)). The assessments provided a more complete understanding of the product and benefits.

Example Product 3 used in the couple study had pigment added to the latex for the partial dip region to highlight the tip portion only and leave the shaft portion unpigmented. Commercial Product 1 did not have pigment in neither the tip nor shaft portions. The appearance of the tip region of Example Product 3 and the type of pigment used were well liked by 72% of the questionnaire respondents. Of the questionnaire respondents that preferred Example Product 3 over Commercial Product 1, multiple respondents cited the color of the tip region as one of the reasons for their preference. The color was described as “fun,” “unique,” “vibrant,” and “exciting.” This was reflected in the qualitative interviews where the tip region was found to be aesthetically pleasing and engaging. The pigment addition was seen as “playful” and “fun,” adding an element of excitement to the product. The unique aesthetics also gave respondents the perception it would offer an enhanced experience (similar to commercial condoms that deliver tingling and warming sensations). Interestingly, the pigment was also noted to mask seminal fluid, making it less visually unappealing after use. Overall, the pigmented tip contributed to a positive user experience.

Example Product 3 used in the couple study had a (T/S) ratio of 1.6 and was tested against the Commercial Product 1 that had an average (T/S) ratio of 1.2. Numerically, more male respondents reported that Example Product 3 provided a more pleasurable orgasm. Based on the variation in the (T/S) ratio, a distinct preference between the tested condoms was expressed due to the unique sensory experienced. The thickened latex of Example Product 3 may have provided a more rigid region of the design and the thinner latex may provide a softer region which may move during intercourse and continually stimulate the glans and shaft. Some of the male panelists also noticed a prolonged duration of intercourse when using Example Product 3. In those instances, the delay in orgasm may have been due physically blocking of nerves along the glans and shaft due to the thickened tip portion. Female respondents noted a sensation of closeness when using Example Product 3. The sensation was described as a ‘warming’ or a ‘closeness’ akin to skin-on-skin, with some respondents articulating a feeling of warmth from their partner's penis during intercourse. This feature was unexpected from female panelists as the standard condoms having a (T/S) ratio of 1.2 were not found to have provided this benefit. Several female panelists described the experience with Example Product 3 as a blended pleasure of different sensations across the condom (tip and shaft) as well as both partners experiencing a shared pleasure. The thinner shaft portion of the condom of Example Product 3 may have provided heightened sensation of simulated body heat along the profile. This feeling of body heat translated to a closer experience and connection between themselves, and partner based on the female respondent's feedback. Furthermore, both male and female respondents reported qualitatively that Example Product 3 was easier to don and remove, and it was significantly more “easy to unroll.” Having a distinctly thickened tip portion of the condom and thinner shaft portion of the condom may have allowed easier donning and removal via a more prominent tip for visual orientation and grasping for disposal.

1. A condom, comprising:
an elongated tube defined by an outer wall comprising an elastomeric material, the elongated tube having a closed end and an open end, the elongated tube having a tip portion that extends a distance from the closed end toward the open end and a shaft portion that extends from the open end to the tip portion;
wherein the outer wall in the tip portion has a tip portion thickness, the outer wall in the shaft portion has a shaft portion thickness, and a ratio of the tip portion thickness to the shaft portion thickness (T/S) is about 1.5 or greater.

2. The condom of claim 1, wherein the ratio of the tip portion thickness to the shaft portion thickness (T/S) is about 1.6 or greater.

3. The condom of claim 1, wherein the ratio of the tip portion thickness to the shaft portion thickness (T/S) is about 1.6 to about 3.8.

4. The condom of claim 1, wherein the tip portion thickness ranges from about 75 μm to about 150 μm.

5. The condom of claim 1, wherein the tip portion thickness ranges from about 100 μm to about 150 μm.

6. The condom of claim 1, wherein the tip portion thickness ranges from about 125 μm to about 150 μm.

7. The condom of claim 1, wherein the shaft portion thickness ranges from about 40 μm to about 95 μm.

8. The condom of claim 1, wherein a number of layers defining the outer wall in the shaft portion is a whole number (n) of layers of the elastomeric material, and the number of layers defining the outer wall in the tip portion is a whole number (x) of the same elastomeric material or a different elastomeric material, wherein x is greater than n.

9. The condom of claim 8, wherein a sum of n and x is 2 or greater.

10. The condom of claim 9, wherein a sum of n and x ranges from 3 to 5.

11. The condom of claim 1, wherein a distance the tip portion extends from the closed end is about 5% to about 50% of a total length of the condom.

12. The condom of claim 11, wherein the distance is about 10% to about 25% of the total length of the condom.

13. The condom of claim 1, wherein the outer wall of the elongated tube comprises at least one full-length layer and at least one partial-length layer.

14. The condom of claim 13, wherein,
the at least one full-length layer has a length of at least 180 mm, and
the at least one partial-length layer has a length ranging from about 10 mm to about 90 mm.

15. The condom of claim 13, wherein the outer wall of the elongated tube comprises a first layer, a second layer, and a third layer,
wherein the first layer is a full-length layer, the second layer is a partial-length layer, and the third layer is a full-length layer.

16. The condom of claim 13, wherein the outer wall of the elongated tube comprises a first layer a second layer, and a third layer,
wherein the first layer is a partial-length layer, the second layer is a full-length layer, and the third layer is a full-length layer.

17. The condom of claim 13, wherein the outer wall of the elongated tube comprises a first layer a second layer, and a third layer,
wherein the first layer is a full-length layer, the second layer is a full-length layer, and the third layer is a partial-length layer.

18. The condom of claim 1, wherein the tip portion is pigmented, and the shaft portion is unpigmented.

19. The condom of claim 18, wherein the tip portion is pigmented with at least one pigment selected from the group consisting of red, orange, yellow, green, blue, indigo, and violet pigments.
